(19) United States
(12) Patent Application Publication
(10) Pub. No.: US 2022/0199224 A1
(43) Pub. Date: Jun. 23, 2022

(54) DE-ESCALATING SITUATIONS

(71) Applicant: International Business Machines Corporation, Armonk, NY (US)

(72) Inventors: Ugo Ivan Orellana Gonzalez, Westlake Village, CA (US); Jo Ann Harris Hill, Coppell, TX (US); Joseph Douglas Harvey, Binghamton, NY (US); Christopher F. Ackermann, Fairfax, VA (US)

(21) Appl. No.: 17/128,251

(22) Filed: Dec. 21, 2020

(51) Int. Cl. G16H 20/70; G16H 40/63

(52) U.S. Cl. CPC G16H 20/70; G16H 40/63

(57) ABSTRACT

An interaction involving two or more users is identified based on at least one biometric value of a biometric attribute of one user of the two or more users exceeding a threshold value of the biometric attribute. A determination is made that the identified interaction is a stressful interaction for the one user. At least one de-escalation recommendation meant to reduce the intensity of the determined stressful interaction is transmitted to the one user.

### BACKGROUND

The present invention relates generally to the field of wearable devices, and more particularly to providing for identifying a stressful situation or interaction via a wearable device and de-escalating said situation.

A wearable device is a smart, electronic device (i.e., an electronic device with one or more micro-controllers) that is worn on or close to the skin. Wearable devices are able to detect, analyze, and transmit information concerning, for example, body signals such as vital signs, and/or ambient data, and which allow in some cases immediate biofeedback to the wearer or someone monitoring the wearer and the output of the wearable devices. One of the simpler wearable devices is an activity or fitness tracker which can track, among other parameters, distances walked or run, calorie intake, heart rate, and blood pressure. Other examples of wearable technology includes, but are not limited to, cameras which provide both audio and video information, smartwatches, devices that collect electroencephalogram (EEG) and electrocardiogram (ECG) data, a wearable sonar band that is able to identify objects in the dark, and a global positioning system (GPS) device for determining location of the wearer.

### SUMMARY OF THE INVENTION

Embodiments of the present invention include an approach for providing for identifying a stressful interaction via a wearable device and deescalating said situation. In one embodiment, an interaction involving two or more users is identified based on at least one biometric value of a biometric attribute of one user of the two or more users exceeding a threshold value of the biometric attribute. A determination is made that the identified interaction is a stressful interaction for the one user. At least one de-escalation recommendation meant to reduce the intensity of the determined stressful interaction is transmitted to the one user.

### BRIEF DESCRIPTION OF THE DRAWINGS

FIG. 1 depicts a functional block diagram illustrating a computing environment which includes a de-escalation program, in accordance with an embodiment of the present invention;

FIG. 2 is a flowchart depicting operational steps of a program for providing for identifying a stressful situation via a wearable device and de-escalating said situation, on a computing device within the computing environment of FIG. 1, in accordance with an embodiment of the present invention; and

FIG. 3 depicts a block diagram of components of a computing device executing a de-escalation program within the computing environment of FIG. 1, in accordance with an embodiment of the present invention.

### DETAILED DESCRIPTION

Embodiments of the present invention recognize that any stressful situation needs to be resolved quickly and peacefully before it gets out-of-hand. For example, a simple disagreement between two people should be able to be resolved peacefully by an intermediary. The disagreement is often just between the two people involved but sometimes, the intermediary becomes involved. A tool is needed that is able to quickly identify a situation that that may get out-of-hand. This tool can be implemented through the adoption of wearable devices which are capable of monitoring certain biometric information of the person wearing said devices. Examples of the biometric information includes, but is not limited to, pulse (i.e., heart rate), blood pressure, skin temperature, perspiration (i.e., sweat) rate including the electrolytes and metabolites within the sweat, electrocardiogram data, and electroencephalogram data. Normal ranges of the user for these biometrics can be determined and a stressful situation can be identified based on changes to the biometrics. Through the utilization of one or more of these wearable devices that can monitor these biometrics, a situation that may get out-of-hand can be identified, and de-escalation instructions can be provided to the user associated with the one or more wearable devices.

Embodiments of the present invention recognize that there may be a method, computer program product, and computer system for providing for identifying a stressful situation via at least one wearable device and deescalating said situation. In this document, ‘situation’ and ‘interaction’ are used interchangeably. The method, computer program product, and computer system improve public safety by assisting an intermediary who is utilizing a wearable device in the quick resolution of a stressful situation. In the context of this document, a stressful is one which causes one or more biometrics of a user of one or more wearable devices to increase beyond threshold levels associated with baseline levels of the one or more biometrics and the safety of a group of people may require the resolution of the stressful situation. The wearable devices are capable of monitoring certain biometric information of the person wearing said devices. Examples of the biometric information includes, but is not limited to, heart rate, blood pressure, temperature, and perspiration (i.e., sweat) rate including the electrolyte and metabolite levels within the sweat. Normal ranges of the user for these biometrics can be determined and a stressful situation can be identified based on changes to the biometrics. Based on the biometric data of the user, and other data (e.g., from a body camera), instructions can be provided to the user in real-time to aid in the quick resolution of the stressful situation.

In an embodiment, a set of initial biometric data is received from each user (i.e., person) in a set of users (i.e., people). In the embodiment, each user is identified, and an association is made between each identified user and their respective set of initial biometric data. Further in the embodiment, baseline values for each biometric value of each user in the set of biometric data of each user are determined. Further yet in the embodiment, the set of biometrics of each user is monitored in real-time. Further yet in the embodiment, a determination is made whether one or more biometric values of a user exceeds a threshold above the baseline value of the biometric value for the user. Further yet in the embodiment, in response to determining that a threshold value of a biometric is exceeded, communication is initiated with the user from a remote facility. Further yet in the embodiment, the current situation is identified. Further yet in the embodiment, de-escalation recommendations (i.e., instructions) are transmitted to the user. Further yet in the embodiment, resources to be utilized are suggested to the user. Further yet in the embodiment, a determination is made whether the biometrics of the user have returned to normal. Further yet in the embodiment, in response to determining that the biometrics of the user have returned to normal, communication ends.

References in the specification to “one embodiment”, “an embodiment”, “an example embodiment”, etc., indicate that the embodiment described may include a particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The present invention will now be described in detail with reference to the Figures.

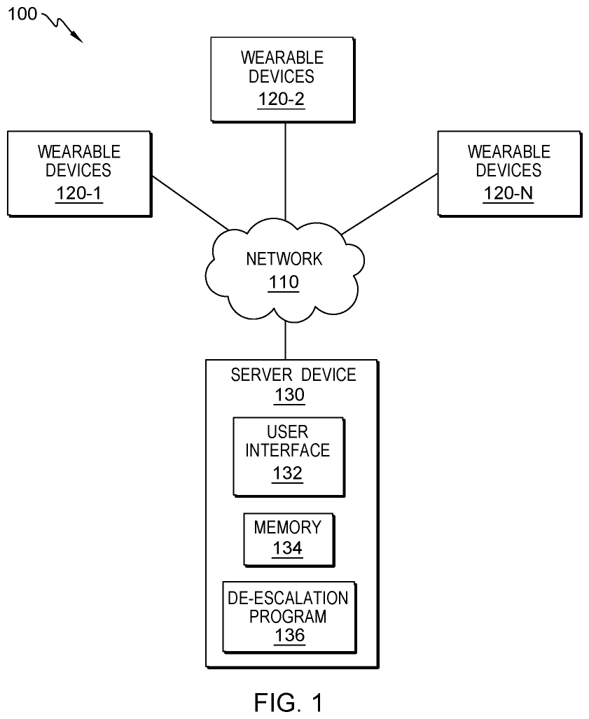

FIG. 1 is a functional block diagram illustrating a computing environment, generally designated 100, in accordance with one embodiment of the present invention. FIG. 1 provides only an illustration of one implementation of the present invention and does not imply any limitations with regard to the systems and environments in which different embodiments may be implemented. Many modifications to the depicted embodiment may be made by those skilled in the art without departing from the scope of the invention as recited by the claims.

In an embodiment, computing environment 100 includes wearable devices 120-1, wearable devices 120-2, wearable devices 120-N, and server device 130 interconnected by network 110. In example embodiments, computing environment 100 includes other computing devices (not shown in FIG. 1) such as additional wearable technology, cell phones, smartphones, phablets, tablet computers, laptop computers, desktop computers, other computer servers, or any other computer system known in the art, interconnected with client device 120, and server device 130 over network 110.

In embodiments of the present invention, wearable devices 120-1, wearable devices 120-2, wearable devices 120-N, and server device 130 are connected to network 110, which enables wearable devices 120-1, wearable devices 120-2, wearable devices 120-N, and server device 130 to access other computing devices and/or data not directly stored on wearable devices 120-1, wearable devices 120-2, wearable devices 120-N, and server device 130. Network 110 may be, for example, a short-range, low power wireless connection, a local area network (LAN), a telecommunications network, a wide area network (WAN) such as the Internet, or any combination of the four, and include wired, wireless, or fiber optic connections. Network 110 includes one or more wired and/or wireless networks that are capable of receiving and transmitting data, voice, and/or video signals, including multimedia signals that include voice, data, and video information. In general, network 110 is any combination of connections and protocols that will support communications between wearable devices 120-1, wearable devices 120-2, wearable devices 120-N, and server device 130, and any other computing devices (not shown in FIG. 1) connected to network 110, in accordance with embodiments of the present invention.

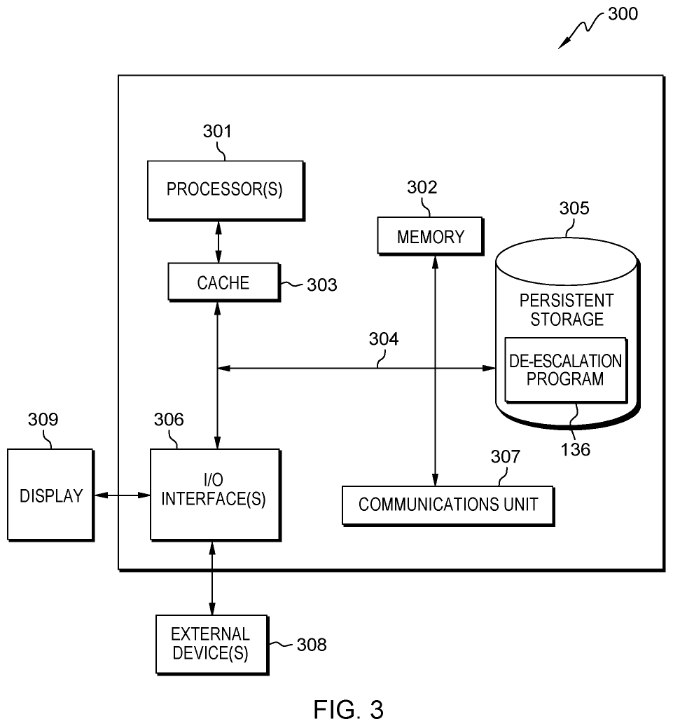

According to embodiments of the present invention, wearable devices 120-1, wearable devices 120-2, and wearable devices 120-N may be one or more miniature electronic devices that may be worn by the bearer (i.e., user) under, with, or on top of clothing, as well as in or connected to glasses, hats, or other accessories. Wearable technologies are especially useful for applications that require more complex computational support than merely hardware coded logics. In an embodiment, wearable devices 120-1 are a set of wearable devices worn by a first person, wearable devices 120-1 are another set of wearable devices worn by a second person, and wearable devices 120-N are yet another set of wearable devices worn by the ‘N^{th}’ person. For ease of reading, wearable devices 120-N will refer to any instance of wearable devices 120-1, wearable devices 120-2, and wearable devices 120-N in this document. In one embodiment, wearable devices 120-N may be in the form of a head mounted display. The head mounted display may take the form-factor of a pair of glasses. Wearable devices 120-N may also be in the form of a smartwatch or a smart tattoo. According to embodiments, wearable devices 120-N can be a standalone computing device, a management server, a web server, a mobile computing device, or any other electronic device or computing system capable of receiving, transmitting, and processing data. In other embodiments, wearable devices 120-N can represent computing systems utilizing multiple computers as a server system, such as in a cloud computing environment. In certain embodiments, wearable devices 120-N represents a computer system utilizing clustered computers and components (e.g., database server computers, application server computers, etc.) that act as a single pool of seamless resources when accessed by elements of computing environment 100. In general, wearable devices 120-N is representative of any electronic device or combination of electronic devices capable of executing computer readable program instructions and collecting biometric information associated with the wearer (e.g., pulse, blood pressure, skin temperature, etc.). In an embodiment, computing environment 100 includes any number of wearable devices 120-N. Wearable devices 120-N may include internal and external hardware components as depicted and described in further detail with respect to FIG. 3, in accordance with embodiments of the present invention.

In an embodiment, server device 130 may be one of a laptop, tablet, or netbook personal computer (PC), a desktop computer, a personal digital assistant (PDA), a smartphone, a standard cell phone, a smartwatch or any other wearable technology, or any other hand-held, programmable electronic device capable of communicating with any other computing device within computing environment 100. According to embodiments, server device 130 can be a standalone computing device, a management server, a web server, a mobile computing device, or any other electronic device or computing system capable of receiving, transmitting, and processing data. In other embodiments, server device 130 can represent computing systems utilizing multiple computers as a server system, such as in a cloud computing environment. In certain embodiments, server device 130 represents a computer system utilizing clustered computers and components (e.g., database server computers, application server computers, etc.) that act as a single pool of seamless resources when accessed by elements of computing environment 100. In general, server device 130 is representative of any electronic device or combination of electronic devices capable of executing computer readable program instructions. In an embodiment, computing environment 100 includes any number of server device 130. Server device 130 may include internal and external hardware components as depicted and described in further detail with respect to FIG. 3, in accordance with embodiments of the present invention. In an embodiment, server device 130 also includes user interface (UI) 132, memory 134, and de-escalation program 136.

According to an embodiment, UI 132 provides an interface between a user of server device 130, wearable device 120-N, and de-escalation program 136. UI 132 may be a graphical user interface (GUI) or a web user interface (WUI) and can display text, documents, web browser windows, user options, application interfaces, and instructions for operation, and include the information (such as graphic, text, and sound) that a program presents to a user and the control sequences the user employs to control the program. UI 132 may also be mobile application software that provides an interface between server device 130, wearable devices 120-N, and de-escalation program 136. Mobile application software, or an “app,” is a computer program designed to run on smartphones, tablet computers and other mobile devices. UI 132 enables a user of server device 130 to interact with wearable devices 120-N, de-escalation program 136, any other programs and applications included on server device 130 (not shown in FIG. 1), and any other computing devices (not shown in FIG. 1).

In an embodiment, memory 134 is storage that is written to and/or read by wearable devices 120-N, de-escalation program 136, and any other programs and applications on wearable devices 120-N and server device 130. In one embodiment, memory 134 resides on server device 130. In other embodiments, memory 134 resides on wearable devices 120-N, on any other device (not shown in FIG. 1) in computing environment 100, in cloud storage, or on another computing device accessible via network 110. In yet another embodiment, memory 134 represents multiple storage devices within server device 130. Memory 134 may be implemented using any volatile or non-volatile storage media for storing information, as known in the art. For example, memory 134 may be implemented with a tape library, optical library, one or more independent hard disk drives, multiple hard disk drives in a redundant array of independent disks (RAID), solid-state drives (SSD), or random-access memory (RAM). Similarly, memory 134 may be implemented with any suitable storage architecture known in the art, such as a relational database, an object-oriented database, or one or more tables. In an embodiment of the present invention, wearable devices 120-N, de-escalation program 136, and any other programs and applications (not shown in FIG. 1) operating on server device 130 may store, read, modify, or write data to memory 134. In an embodiment of the present invention, data stored to memory 134 includes, but is not limited to, a set of biometrics data for each person associated with wearable devices 120-N.

According to an embodiment of the present invention, de-escalation program 136 is a program, a subprogram of a larger program, an application, a plurality of applications, or mobile application software, which functions to provide for identifying a stressful situation via a wearable device and de-escalating said situation. In the context of this document, a stressful situation is one which causes one or more biometrics of a user of one or more wearable devices to increase beyond threshold levels associated with baseline levels of the one or more biometrics and the safety of one or more people (i.e., a group of people) may require the resolution of the stressful situation. A program is a sequence of instructions written to perform a specific task. In an embodiment, de-escalation program 136 runs independently. In other embodiments, de-escalation program 136 depends on system software and/or other programs (not shown in FIG. 1) to execute. According to an embodiment, de-escalation program 136 is a cognitive system based on artificial intelligence utilizing machine learning and deep learning which processes data from one or more biometric wearable devices worn by a user and determines that a situation is stressful when biometric data of a user exceeds a threshold delta above the normal value of the biometric data. When de-escalation program 136 identifies the stressful situation based on the biometric data, de-escalation program 136 automatically initiates communication with the user and provides one or more recommendations (i.e., instructions) to the user to resolve the stressful situation. In one embodiment, de-escalation program 136 functions as a stand-alone program residing on server device 130. In another embodiment, de-escalation program 136 works in conjunction with other programs, applications, etc., found in computing environment 100. In yet another embodiment, de-escalation program 136 resides on other computing devices such as wearable devices 120-N in computing environment 100, which are interconnected to server device 130 via network 110.

According to an embodiment, de-escalation program 136 receives a set of initial biometric data for a user. In the embodiment, de-escalation program 136 identifies the user and associates the set of initial biometric data with the identified user. Further in the embodiment, de-escalation program 136 determines baseline values for each biometric value in the set of initial biometrics data. Further yet in the embodiment, de-escalation program 136 monitors the real-time biometric values of the user. Further yet in the embodiment, de-escalation program 136 determines whether any real-time, individual biometric value of the user exceeds the baseline value of said biometric by a threshold amount. Further yet in the embodiment, responsive to determining an individual biometric value of the user does exceed the baseline value of said biometric by a threshold amount, de-escalation program 136 initiates communication between the user and a remote facility. Further yet in the embodiment, de-escalation program 136 identifies the current situation of the user. Further yet in the embodiment, de-escalation program 136 transmits one or more recommendations to the user aimed at de-escalating the situation. Further yet in the embodiment, de-escalation program 136 suggests one or more appropriate resources to be utilized by the user in the situation. Further yet in the embodiment, de-escalation program 136 determines whether the biometric values of the user have returned to normal. Further yet in the embodiment, responsive to determining that the biometric values of the user have returned to normal, de-escalation program 136 ends communication between the user and the remote facility.

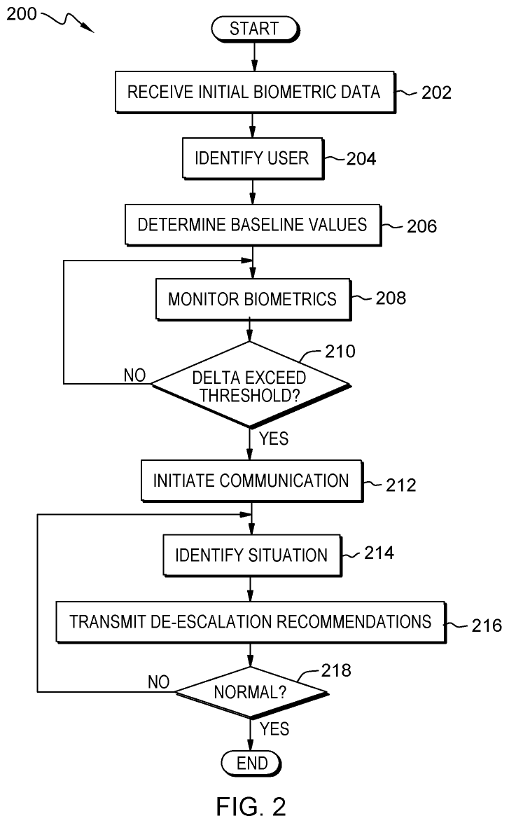

FIG. 2 is a flowchart of workflow 200 depicting operational steps for providing for identifying a stressful situation via a wearable device and de-escalating said situation. In one embodiment, the method of workflow 200 is performed by de-escalation program 136. In an alternative embodiment, the method of workflow 200 is performed by any other program in computing environment 100 working with de-escalation program 136. In an embodiment, a user of server device 130 invokes workflow 200 upon accessing de-escalation program 136. In another embodiment, de-escalation program 136 is invoked by a user (i.e., wearer) of wearable devices 120-N. In yet another embodiment, workflow 200 is dynamically invoked upon a biometric value of a user exceeding a threshold delta above a baseline value of said biometric.

In an embodiment, de-escalation program 136 receives initial biometric data (step 202). In other words, de-escalation program 136 receives a set of initial biometric data for one or more users. According to an embodiment, the set of initial biometric data is received from one or more wearable devices associated with the one or more users. Each user of the one or more users will have an individual set of initial biometric data. Examples of biometric data within the initial biometric data includes, but are not limited to, heart rate (i.e., pulse), blood pressure, temperature, and sweat level. According to the embodiment, de-escalation program 136 stores the received initial biometric data to a memory. In an embodiment, de-escalation program 136 receives over network 110 initial biometric data from wearable devices 120-N from one or more users and stores the initial biometric data to memory 134 on server device 130. For example, a program on a server located in a remote monitoring facility receives initial biometric data over a mobile data network for two users. The initial biometric data for each user includes their pulse, blood pressure, and skin temperature over a three week period of time with the parameters measured once per hour for each user. All of the received biometric data is stored to a database on the server.

According to an embodiment of the present invention, de-escalation program 136 identifies users (step 204). In other words, de-escalation program 136 uses any known techniques to identify each user. In an embodiment, de-escalation program 136 may analyze the received initial biometric data to identify the user associated with the biometric data. De-escalation program 136 may also use a GPS location of where the initial biometric data originates and identify a user based on said location. Once the user is identified, de-escalation program 136 creates an association between each user and their respective initial biometric data. In the embodiment, de-escalation program 136 determines whether a user has opted-in to being identified and based on said determination, only identifies opted-in users. In the embodiment, de-escalation program 136 may utilize various accessible data sources that may include personal data, content, or information the one or more users wish not to be processed. Personal data includes personally identifying information or sensitive personal information as well as user information, such as tracking or geolocation information. Processing refers to any operation, automated or unautomated, or set of operations such as collecting, recording, organizing, structuring, storing, adapting, altering, retrieving, consulting, using, disclosing by transmission, dissemination, or otherwise making available, combining, restricting, erasing, or destroying personal data. Opting-in to use de-escalation program 136 enables the authorized and secure processing of personal data. De-escalation program 136 provides informed consent, with notice of the collection of personal data, allowing the one or more users to opt-in or opt-out of processing personal data. Consent can take several forms. Opt-in consent can impose on a user to take an affirmative action before personal data is processed. Alternatively, opt-out consent can impose on the user to take an affirmative action to prevent the processing of personal data before personal data is processed. De-escalation program 136 provides information regarding personal data and the nature (e.g., type, scope, purpose, duration, etc.) of the processing. De-escalation program 136 provides the one or more users with copies of stored personal data. Further, de-escalation program 136 allows for the correction or completion of incorrect or incomplete personal data and also allows for the immediate deletion of personal data. According to an embodiment, de-escalation program 136 identifies the one or more users of the initial biometric data based on meta-data included within the initial biometric data; further, de-escalation program 136 associates each identified user with their respective initial biometric data. For example, the program on the server in the remote monitoring facility identifies the two users as ‘Dan’ and ‘Jon’ based on the GPS locations associated with the wearable devices used by ‘Dan’ and ‘Jon’ and their respective home addresses which are stored to a database accessible by the program. A first set of initial biometric data is associated with ‘Dan’ while a second set is associated with Jon′.

In an embodiment, de-escalation program 136 determines baseline values (step 206). In other words, de-escalation program 136 analyzes the received initial biometric data to determine a baseline value for each parameter (i.e., attribute) included in said data. According to an embodiment, de-escalation program 136 determines the baseline values by retrieving the initial biometrics data from memory and averaging each of the pulse, blood pressure, and skin temperature values for each user in the one or more users over the three week time period. This determines, for each user, a baseline pulse value, a baseline blood pressure, and a baseline skin temperature. These baseline values are stored to memory. In an embodiment, de-escalation program 136 determines the baseline biometric values for each user in the one or more users based on the received initial biometric data; said baseline values for each user are stored to memory 134 on server 130. For example, the program on the server in the remote facility determines the following baseline biometric values for ‘Dan’—baseline pulse of ‘55’ beats per minute, baseline blood pressure of ‘115’ systolic and ‘74’ diastolic (i.e., ‘115/74’), and baseline skin temperature of ‘89’ degrees Fahrenheit (F). For Jon′, the determined baseline biometric values are as follows—baseline pulse of ‘50’, baseline blood pressure of ‘112/70’, and baseline skin temperature of ‘90’ degrees F.

According to an embodiment, de-escalation program 136 monitors biometrics (step 208). In other words, de-escalation program 136 monitors the various biometrics of each user of the one or more users in real-time. In an embodiment, some of the biometrics are sampled continuously (e.g., pulse and skin temperature) while other biometrics are sampled on a predetermined frequency (e.g., blood pressure is taken every ten minutes). The monitored biometric values for each user are stored with other data belonging to the same user. De-escalation program 136 monitors the biometrics via data collected from wearable devices worn by each user. According to an embodiment, de-escalation program 136 monitors the biometrics of each user of the one or more users by receiving data from wearable devices 120-N over network 110; as the data is received, de-escalation program 136 stores the data to memory 134 on server device 130. For example, pulse and skin temperature readings of ‘Dan’ and ‘Jon’ are taken continuously and are sent to the program on the server located in the remote facility for storage while blood pressure readings are taken from ‘Dan’ and ‘Jon’ once every five minutes and are sent to the same program for storage.

In an embodiment, de-escalation program 136 determines whether a delta exceeds a threshold (decision step 210). In other words, de-escalation program 136 determines whether a delta between a current biometric value and an associated baseline biometric value exceeds a threshold value for at least one user of the one or more users. This determination is made for each user in the one or more users and for each biometric attribute in the initial biometric data. According to an embodiment, the threshold value is at least one of (i) a percent increase (e.g., a pulse should not increase more than twenty-five percent), (ii) a maximum value (e.g., a blood pressure should not exceed a value of ‘140/90’), and (iii) a numerical increase in value (e.g., a skin temperature should not increase by more than ‘10’ degrees F. For example, if ‘Dan's’ current pulse is ‘72’, that value is a thirty one percent increase over the associated baseline value of ‘55’. The standard formula to calculate the percent change is current value minus baseline value, divide that result by baseline value, and multiply that result by one hundred (i.e., (72-55)/55*100=30.9 percent). A thirty percent increase would exceed the threshold of a twenty-five percent increase for a pulse reading. The increase in ‘Dan's’ pulse is caused by a stressful situation which ‘Dan’ and ‘Jon’ encountered. In one embodiment (decision step 210, NO branch), de-escalation program 136 determines that there is not a delta which exceeds a threshold between a current biometric value and its associated baseline value for at least one of the one or more users; therefore, de-escalation program 136 returns to step 208 to continue monitoring biometrics. In the embodiment (decision step 210, YES branch), de-escalation program 136 determines that there is a delta which exceeds a threshold between a current biometric value and its associated baseline value for at least one of the one or more users; therefore, de-escalation program 136 proceeds to step 212 to initiate communication.

In another embodiment, de-escalation program 136 can help to identify life-threatening situations of the user of wearable devices 120-N. For example, if the user is stricken by a heart attack and the user's heart stops, de-escalation program 136 would identify that the user's biometrics exceed a threshold delta. In the example, if communication cannot be established (please refer to the following paragraph for details) between the user and a remote monitoring facility, assistance can be summoned for the user automatically by de-escalation program 136.

According to an embodiment, de-escalation program 136 initiates communication (step 212). In other words, responsive to determining that a delta between a current biometric value and its associated baseline value exceeds a threshold value for at least one user of the one or more users, de-escalation program 136 initiates communication between the at least one user and the remote monitoring facility. In an embodiment, de-escalation program 136 initiates at least one of audio communication and audio/video communication using any communication technologies known in the art. The communication is initiated between the at least one user and the remote monitoring facility so that de-escalation program 136 and/or personnel located at the remote monitoring facility can communicate in real-time with the at least one user. The initiated communication allows for fast analysis of the situation and instant transfer of information (e.g., de-escalation instructions) between the at least one user and the personnel at the remote monitoring facility. According to an embodiment, de-escalation program 136 initiates communication over network 110 with the at least one user associated with wearable devices 120-N whose current biometric value(s) exceed a threshold value over the associated baseline values. For example, the program on the server in the remote monitoring facility initiates audio communication with ‘Dan’ via a cellular network based on ‘Dan's’ pulse exceeding a threshold value over the baseline value of ‘Dan's’ pulse. Because ‘Jon’ is with ‘Dan’, communication is also initiated with ‘Jon’ even though the biometric values for ‘Jon’ have not exceeded threshold values (i.e., ‘Jon’ is better able to remain calm in a stressful situation).

In an embodiment, de-escalation program 136 identifies a situation (step 214). In other words, de-escalation program 136 uses various techniques known in the art to identify the situation which caused the biometric value of the user to exceed a threshold. According to an embodiment of the present invention, the identified situation may or may not be a stressful situation. According to the embodiment, de-escalation program 136 may, based on audio input from the initiated communication, use word recognition, natural language processing, and machine learning to ingest the audio input, analyze said input, including tone and volume of the audio, and identify the situation. Further, according to the embodiment, de-escalation program 136 may, based on audio/video input from the initiated communication, use object detection and object recognition techniques along with the previously described audio techniques to ingest the audio/video input, analyze said input, and identify the situation. De-escalation program 136 may compare the analyzed input with a database of known situations, both stressful and non-stressful, and associated words and objects to aid in the identification of the situation. In an embodiment, de-escalation program 136 identifies the situation of the at least one user based on input from the initiated communication received via network 110 and data received from wearable devices 120-N. For example, the program on the server in the remote monitoring facility identifies the situation of two people having an argument based on the communication between ‘Dan’, ‘Jon’, and the remote monitoring facility which is received via the cellular network.

According to an embodiment of the present invention, de-escalation program 136 transmits de-escalation recommendations (step 216). In other words, based on identifying the situation which caused the increased biometric value, de-escalation program 136 dynamically transmits at least one de-escalation recommendation to the at least one user. In one embodiment, de-escalation program 136 dynamically generates the at least one de-escalation recommendation. In another embodiment, de-escalation program 136 receives the at least one de-escalation recommendation from a person monitoring the situation. In yet another embodiment, de-escalation program 136 retrieves the at least one de-escalation recommendation from a database of recommendations which are associated with various situations. In an embodiment, the at least one de-escalation recommendation is meant to reduce the intensity of the identified situation. Example recommendations include, but are not limited to, recommending that the at least one user remain calm during the situation; recommending that the at least one user show respect to the involved parties; recommending that the at least one user utilize learned skills such as conflict resolution, active listening, and patience (i.e., stalling for time); recommending that the at least one user request a skilled mediator or negotiator; recommending that the at least one user separate the involved parties for a period of time; recommending that the at least one user requests additional personnel including a canine to assist in resolving the situation; and recommending that the at least one user set up a perimeter around the identified situation and/or restrain the involved parties for a period of time. Based on continued communication input from the at least one user, de-escalation program 136 continues to monitor the on-going situation and make appropriate recommendations as time goes on. According to an embodiment, de-escalation program 136 transmits a de-escalation recommendation via network 110 to the at least one user associated with wearable devices 120-N whose biometric values have exceeded a threshold value of the associated baseline biometric values. For example, the program on the server at the remote monitoring facility recommends to ‘Dan’ and Jon′ that ‘Dan’ request canine assistance as it has been demonstrated that the presence of a canine can cause stressed people to calm down and thus bring an end the argument between the two people. The program further recommends to ‘Dan’ and Jon′ that ‘Jon’ take the lead in trying to resolve the stressful situation as based on the available biometrics data, Jon′ is more relaxed than ‘Dan’.

In an embodiment, de-escalation program 136 determines whether the biometric attribute has returned to normal (i.e., baseline levels) (decision step 218). In other words, de-escalation program 136 determines whether the biometric attribute(s) of the at least one user associated with wearable devices 120-N have returned to baseline levels and no longer exceed threshold levels indicating that the stressful situation has been resolved. According to an embodiment, the determination is made by data collected by de-escalation program 136 from the continuous monitoring of wearable devices 120-N. In one embodiment (decision step 218, NO branch), de-escalation program 136 determines that the biometric attribute(s) of the at least one user have not returned to baseline levels; therefore, de-escalation program 136 returns to step 214 to identify the situation (i.e., the situation may be the same or the situation may have changed for better or worse). In the embodiment (decision step 218, YES branch), de-escalation program 136 determines that the biometric attribute(s) of the at least one user have returned to baseline levels; therefore, de-escalation program 136 ends the communication and ends processing.

FIG. 3 depicts computer system 300, which is an example of a system that includes de-escalation program 136. Computer system 300 includes processors 301, cache 303, memory 302, persistent storage 305, communications unit 307, input/output (I/O) interface(s) 306 and communications fabric 304. Communications fabric 304 provides communications between cache 303, memory 302, persistent storage 305, communications unit 307, and input/output (I/O) interface(s) 306. Communications fabric 304 can be implemented with any architecture designed for passing data and/or control information between processors (such as microprocessors, communications and network processors, etc.), system memory, peripheral devices, and any other hardware components within a system. For example, communications fabric 304 can be implemented with one or more buses or a crossbar switch.

Memory 302 and persistent storage 305 are computer readable storage media. In this embodiment, memory 302 includes random access memory (RAM). In general, memory 302 can include any suitable volatile or non-volatile computer readable storage media. Cache 303 is a fast memory that enhances the performance of processors 301 by holding recently accessed data, and data near recently accessed data, from memory 302.

Program instructions and data used to practice embodiments of the present invention may be stored in persistent storage 305 and in memory 302 for execution by one or more of the respective processors 301 via cache 303. In an embodiment, persistent storage 305 includes a magnetic hard disk drive. Alternatively, or in addition to a magnetic hard disk drive, persistent storage 305 can include a solid-state hard drive, a semiconductor storage device, read-only memory (ROM), erasable programmable read-only memory (EPROM), flash memory, or any other computer readable storage media that is capable of storing program instructions or digital information.

The media used by persistent storage 305 may also be removable. For example, a removable hard drive may be used for persistent storage 305. Other examples include optical and magnetic disks, thumb drives, and smart cards that are inserted into a drive for transfer onto another computer readable storage medium that is also part of persistent storage 305.

Communications unit 307, in these examples, provides for communications with other data processing systems or devices. In these examples, communications unit 307 includes one or more network interface cards. Communications unit 307 may provide communications through the use of either or both physical and wireless communications links. Program instructions and data used to practice embodiments of the present invention may be downloaded to persistent storage 305 through communications unit 307.

I/O interface(s) 306 allows for input and output of data with other devices that may be connected to each computer system. For example, I/O interface 306 may provide a connection to external devices 308 such as a keyboard, keypad, a touch screen, and/or some other suitable input device. External devices 308 can also include portable computer readable storage media such as, for example, thumb drives, portable optical or magnetic disks, and memory cards. Software and data used to practice embodiments of the present invention can be stored on such portable computer readable storage media and can be loaded onto persistent storage 305 via I/O interface(s) 306. PO interface(s) 306 also connect to display 309.

Display 309 provides a mechanism to display data to a user and may be, for example, a computer monitor.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

What is claimed is:

1. A method, the method comprising:
identifying, by one or more computer processors, an interaction involving two or more users based on at least one biometric value of a biometric attribute of one user of the two or more users exceeding a threshold value of the biometric attribute;
determining, by one or more computer processors, that the identified interaction is a stressful interaction for the one user; and
transmitting, by one or more computer processors, at least one de-escalation recommendation meant to reduce an intensity of the determined stressful interaction to the one user.

2. The method of claim 1, wherein the stressful interaction is an interaction which causes one or more biometrics monitored by one or more wearable devices of the one user to increase beyond threshold levels associated with baseline levels of the one or more biometrics and safety of a group of users may require a resolution of the stressful interaction.

3. The method of claim 1, wherein the step of determining, by one or more computer processors, that the identified interaction is a stressful interaction for the one user, comprises:
receiving, by one or more computer processors, from one or more wearable devices a set of initial biometric data associated with at least one user;
identifying, by one or more computer processors, the at least one user, wherein the at least one user has opted-in to being identified;
determining, by one or more computer processors, baseline values for each biometric attribute included in a set of initial biometric data of the identified at least one user;
monitoring, by one or more computer processors, real-time biometric values for each biometric attribute included in the set of initial biometric data of the identified at least one user;
responsive to determining that a delta between a current real-time biometric value of at least one biometric attribute of the identified at least one user and an associated baseline value of the at least one biometric value exceeds a threshold value, initiating, by one or more computer processors, communication between the identified at least one user and a monitoring facility;
identifying, by one or more computer processors, the interaction of the identified at least one user based on analyzing the initiated communication between the at least one user and the monitoring facility; and
comparing, by one or more computer processors, the identified interaction with a database of known stressful and non-stressful interactions to determine if the identified interaction is stressful.

4. The method of claim 3, wherein:
the initiated communication includes at least one of audio communication and audio/video communication; and
the analysis of the initiated communication includes one or more of word recognition, natural language processing, machine learning, object detection, and object recognition.

5. The method of claim 1, wherein the biometric attribute of the one user is selected from the group consisting of a pulse, a blood pressure, a skin temperature, a perspiration rate including electrolyte and metabolite levels, an electroencephalogram reading, and an electrocardiogram reading.

6. The method of claim 1, wherein the threshold value of the biometric attribute is at least one of a percentage increase in the biometric value of the biometric attribute, a maximum value for the biometric value of the biometric attribute, and a numerical increase in the biometric value of the biometric attribute.

7. The method of claim 3, further comprising:
responsive to determining that a delta between a current real-time biometric value of at least one biometric attribute of the identified at least one user and an associated baseline value of the at least one biometric value does not exceed a threshold value, continue monitoring, by one or more computer processors, the real-time biometric values for each biometric attribute included in the set of initial biometric data of the identified at least one user.

8. The method of claim 1, further comprising:
responsive to transmitting the at least one de-escalation recommendation meant to reduce the intensity of the determined stressful interaction to the one user, determining, by one or more computer processors, whether the at least one biometric value of the biometric attribute of the one user no longer exceeds the threshold value of the biometric attribute; and
responsive to determining that the at least one biometric value of the biometric attribute of the one user no longer exceeds the threshold value of the biometric attribute, ending, by one or more computer processors, the initiated communication between the one user and the monitoring facility.

9. The method of claim 8, further comprising:
responsive to determining that the at least one biometric value of the biometric attribute of the one user continues to exceed the threshold value of the biometric attribute, identifying, by one or more computer processors, the interaction of the one user, wherein the interaction may be as previously identified or may have changed.

10. A computer program product, the computer program product comprising:
one or more computer readable storage media; and
program instructions stored on the one or more computer readable storage media, the program instructions comprising:
program instructions to identify an interaction involving two or more users based on at least one biometric value of a biometric attribute of one user of the two or more users exceeding a threshold value of the biometric attribute;
program instructions to determine that the identified interaction is a stressful interaction for the one user; and
program instructions to transmit at least one de-escalation recommendation meant to reduce an intensity of the determined stressful interaction to the one user.

11. The computer program product of claim 10, wherein the stressful interaction is an interaction which causes one or more biometrics monitored by one or more wearable devices of the one user to increase beyond threshold levels associated with baseline levels of the one or more biometrics and safety of a group of users may require a resolution of the stressful interaction.

12. The computer program product of claim 10, wherein the program instructions to determine that the identified interaction is a stressful interaction for the one user, comprise:
program instructions to receive from one or more wearable devices a set of initial biometric data associated with at least one user;
program instructions to identify the at least one user, wherein the at least one user has opted-in to being identified;
program instructions to determine baseline values for each biometric attribute included in a set of initial biometric data of the identified at least one user;
program instructions to monitor real-time biometric values for each biometric attribute included in the set of initial biometric data of the identified at least one user;
responsive to determining that a delta between a current real-time biometric value of at least one biometric attribute of the identified at least one user and an associated baseline value of the at least one biometric value exceeds a threshold value, program instructions to initiate communication between the identified at least one user and a monitoring facility;
program instructions to identify the interaction of the identified at least one user based on analyzing the initiated communication between the at least one user and the monitoring facility; and
program instructions to compare the identified interaction with a database of known stressful and non-stressful interactions to determine if the identified interaction is stressful.

13. The computer program product of claim 12, wherein:
the initiated communication includes at least one of audio communication and audio/video communication; and
the analysis of the initiated communication includes one or more of word recognition, natural language processing, machine learning, object detection, and object recognition.

14. The computer program product of claim 10, wherein the biometric attribute of the one user is selected from the group consisting of a pulse, a blood pressure, a skin temperature, a perspiration rate including electrolyte and metabolite levels, an electroencephalogram reading, and an electrocardiogram reading.

15. The computer program product of claim 10, wherein the threshold value of the biometric attribute is at least one of a percentage increase in the biometric value of the biometric attribute, a maximum value for the biometric value of the biometric attribute, and a numerical increase in the biometric value of the biometric attribute.

16. The computer program product of claim 12, further comprising program instructions stored on the one or more computer readable storage media, to:
responsive to determining that a delta between a current real-time biometric value of at least one biometric attribute of the identified at least one user and an associated baseline value of the at least one biometric value does not exceed a threshold value, continue monitoring, by one or more computer processors, the real-time biometric values for each biometric attribute included in the set of initial biometric data of the identified at least one user.

17. The computer program product of claim 10, further comprising program instructions stored on the one or more computer readable storage media, to:
responsive to transmitting the at least one de-escalation recommendation meant to reduce the intensity of the determined stressful interaction to the one user, determining, by one or more computer processors, whether the at least one biometric value of the biometric attribute of the one user no longer exceeds the threshold value of the biometric attribute; and
responsive to determining that the at least one biometric value of the biometric attribute of the one user no longer exceeds the threshold value of the biometric attribute, ending, by one or more computer processors, the initiated communication between the one user and the monitoring facility.

18. The computer program product of claim 17, further comprising program instructions stored on the one or more computer readable storage media, to:
responsive to determining that the at least one biometric value of the biometric attribute of the one user continues to exceed the threshold value of the biometric attribute, identifying, by one or more computer processors, the interaction of the one user, wherein the interaction may be as previously identified or may have changed.

19. A computer system, the computer system comprising:
one or more computer processors;
one or more computer readable storage media; and
program instructions stored on the one or more computer readable storage media for execution by at least one of the one or more computer processors, the program instructions comprising:
program instructions to identify an interaction involving two or more users based on at least one biometric value of a biometric attribute of one user of the two or more users exceeding a threshold value of the biometric attribute;
program instructions to determine that the identified interaction is a stressful interaction for the one user; and
program instructions to transmit at least one de-escalation recommendation meant to reduce an intensity of the determined stressful interaction to the one user.

20. The computer system of claim 19, wherein the stressful interaction is an interaction which causes one or more biometrics monitored by one or more wearable devices of the one user to increase beyond threshold levels associated with baseline levels of the one or more biometrics and safety of a group of users may require a resolution of the stressful interaction.
